(19) United States
(12) Patent Application Publication
(10) Pub. No.: US 2009/0264767 A1
(43) Pub. Date: Oct. 22, 2009

(54) METHOD AND APPARATUS FOR ULTRASONIC IMAGING USING TRANSDUCER ARRAYS

(75) Inventors: Weston Blaine Griffin, Niskayuna, NY (US); Douglas Glenn Wildes, Ballston Lake, NY (US); Warren Lee, Niskayuna, NY (US); Terry Michael Topka, Scotia, NY (US)

Correspondence Address: GENERAL ELECTRIC COMPANY;GLOBAL RESEARCH, PATENT DOCKET RM. BLDG. K1-4A59, NISKAYUNA, NY, 12309, US

(73) Assignee: GENERAL ELECTRIC COMPANY, Schenectady, NY (US)

(21) Appl. No.: 12/106,383

(22) Filed: Apr. 21, 2008

(51) Int. Cl. A61B 8/00

(52) U.S. Cl. 600/459

(57) ABSTRACT

An ultrasonic imaging system and method for acquiring three-dimensional (3D) image data sets are provided. The system comprises a transducer array with a given range of motion adapted to obtain a plurality of 3D image data sets of a region of interest and a processor coupled to the transducer array adapted to receive image data sets from the transducer array and to correct for spatially varying errors induced by motion of the transducer array.

### BACKGROUND

The invention relates generally to ultrasonic imaging systems, and more particularly to two- and three-dimensional ultrasonic imaging using mechanically scanning transducer arrays for acquiring the image data.

Briefly summarized, three-dimensional ultrasonic imaging systems using mechanically scanning transducer arrays refers to various approaches to real-time three-dimensional (“RT3D,” a.k.a. “4D”) ultrasonic imaging, including those that use a catheter-based ultrasound probe. Real time three-dimensional ultrasonic imaging from a unit housed in a catheter offers many advantages for conducting exacting diagnostic and interventional procedures. Accordingly, improvements in this field are expected to offer substantial cost effectiveness and other benefits for medical diagnostics and interventions.

A catheter-based ultrasound probe for real-time three-dimensional imaging generally includes at least one ultrasound transducer array positioned longitudinally along the catheter. The ultrasound transducer array is connected to a drive shaft that moves the array relative to the patient, to generate a plurality of spatially related two-dimensional tomographic images of body structure adjacent the catheter. A control system includes a drive mechanism that may be positioned within the catheter body or may be remotely located from the catheter body. The catheter-based ultrasound probe may include an integral catheter tip that comprises an array of at least one transducer for transmitting ultrasound energy radially outward, and for receiving ultrasound energy. As used herein, the term “radially” may include angles other than 90° to the catheter axis. For instance, a mostly forward-looking 4D probe, would have forward-oriented cone field of view. Imaging proceeds by rotation or oscillation of the array, such as by using micromotor actuators. Some actuators move the array in the circumferential direction, and some actuators move the array axially forward and back. Thus, three-dimensional volumetric images may be obtained by use of this catheter and transducer array assembly.

However, such mechanically scanning transducer arrays introduce inherent errors due to the mechanical motion, such as the positional errors introduced by the mechanical motion of the array that can cause distortion of the rendered image or volume, and jitter in real-time images or volumes.

To create an accurate representation of a given volume based on ultrasound signals, the acquisition system must know the location in the object or patient at which each ultrasound beam or beam set is acquired. With a mechanically scanned ultrasound array, the mechanical system can introduce errors in the actual positioning of the array as compared to the intended, or commanded, position of the array. The positioning errors may cause an ultrasound beam or beam set to be acquired in a different location that originally intended or expected. In this case, the displayed image of the target, rendered based on the intended locations of the beams, may exhibit some geometric distortion.

Additionally, small mechanical probes, e.g. 4D intracardiac echocardiography (ICE) catheters, typically have compliant, or “soft”, low-power drive systems with very repeatable but highly non-linear, asymmetric motion. Imaging during bi-directional motion results in severe image jitter, because the forward images are not aligned with the reverse images.

To use ultrasound to effectively visualize moving anatomy, especially for invasive procedures, it is desirable to have real-time 3D (a.k.a. 4D) images updated significantly faster than the anatomy is moving. Mechanical (moving transducer) 4D ultrasound probes are cost-effective and can be high-performance if the tissue motion is not too fast. In further embodiments, methods to optimize mechanical 4D ultrasound probes, drive systems, and imaging to obtain high quality images with fast update rates are provided. These methods may be particularly applicable to probes for imaging in confined spaces, e.g. invasive probes, e.g. endoscopes, laparoscopes, or catheters. High-quality imaging should be geometrically accurate and stable. Invasive ultrasound probes must be small. Small mechanical systems tend to be low-power and not very stiff. When operated at high speed, for fast imaging, small mechanical ultrasound probes exhibit multiple non-ideal behaviors: hysteresis/backlash; non-linearity; dynamics/modes; etc. These behaviors both distort the apparent geometry of the anatomy and cause poor alignment between successive images of the same anatomy, i.e. image instability. Small probes, e.g. catheters, have very limited space for position sensors, so real-time feedback to correct the non-linearities is not easily achieved.

What is needed is a method and system for 3D ultrasonic imaging using transducer arrays that correct for errors attributed to mechanically scanning transducer arrays.

### BRIEF DESCRIPTION

In a first aspect, provided is an ultrasonic imaging system for acquiring three-dimensional (3D) image data sets comprising a transducer array with a given range of motion adapted to obtain a plurality of 3D image data sets of a region of interest; a processor coupled to the transducer array adapted to receive image data sets from the transducer array and to correct for spatially varying error induced by motion of the transducer array.

In a second aspect, a method for ultrasonic diagnostic imaging using a mechanically moving transducer array is provided and the method comprises the steps of obtaining a plurality of three-dimensional (3D) image data sets along a given range of motion and/or a plurality of firing positions; and, correcting for spatially varying errors induced by motion of the transducer array in order to display 3D image data sets.

### DRAWINGS

These and other features, aspects, and advantages of the present invention will become better understood when the following detailed description is read with reference to the accompanying drawings in which like characters represent like parts throughout the drawings, wherein:

FIG. 1 is a block diagram of an exemplary catheter imaging and therapy system, in accordance with and/or adaptable to utilize aspects of the present apparatus and methods.

FIG. 2 is a side and internal view of an exemplary embodiment of a catheter tip comprising a rotating transducer array.

FIG. 3 is an illustration of a process flow for an embodiment of a method for correcting image data sets during ultrasonic imaging for use in processor of FIG. 1.

FIG. 4 is an illustration of a process flow for an embodiment of a method for correcting image data sets during ultrasonic imaging for use in processor of FIG. 1.

### DETAILED DESCRIPTION

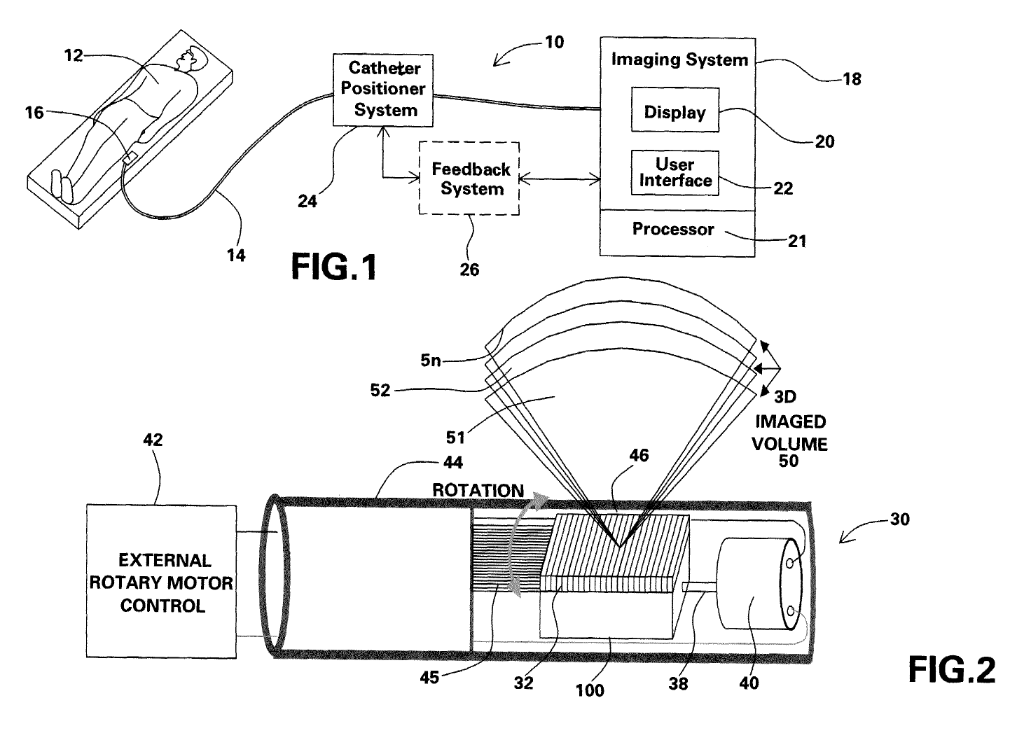

FIG. 1 is a block diagram of an exemplary ultrasound imaging system 10 for use in imaging and providing therapy to one or more regions of interest in accordance with aspects of the present technique. The system 10 may be configured to acquire image data from a patient 12 via a catheter 14. As used herein, “catheter” is broadly used to include conventional catheters, endoscopes, laparoscopes, transducers, probes or devices adapted for imaging as well as adapted for applying therapy. Further, as used herein, “imaging” is broadly used to include two-dimensional imaging, three-dimensional imaging, or preferably, real-time three-dimensional imaging. Further, as used herein, “fluid” may be interpreted broadly to include a liquid or a gel. Reference numeral 16 is representative of a portion of the catheter 14 disposed on or inside the body of the patient 12. This portion 16 may comprise a catheter tip as is disclosed and described in later figures.

In certain embodiments, an imaging orientation of the imaging and therapy catheter 14 may include a forward viewing catheter or a side-viewing catheter. However, a combination of forward viewing and side viewing catheters may also be employed as the catheter 14. Catheter 14 may include a real-time imaging and therapy transducer (not shown). According to aspects of the present technique, the imaging and therapy transducer may include integrated imaging and therapy components. Alternatively, the imaging and therapy transducer may include separate imaging and therapy components. The transducer in an exemplary embodiment is a 64 element one-dimensional (1D) transducer array and will be described further with reference to FIG. 2. It should be noted that although the embodiments illustrated are described in the context of a catheter-based transducer, other types of transducers such as transesophageal transducers or transthoracic transducers are also contemplated.

In accordance with aspects of the present technique, the catheter 14 may be configured to image an anatomical region to facilitate assessing need for therapy in one or more regions of interest within the anatomical region of the patient 12 being imaged. Additionally, the catheter 14 may also be configured to deliver therapy to the identified one or more regions of interest. As used herein, “therapy” is representative of ablation, percutaneous ethanol injection (PEI), cryotherapy, high intensity focused ultrasound (HIFU), and laser-induced thermotherapy. Additionally, “therapy” may also include delivery of tools, such as needles for delivering gene therapy, for example. Additionally, as used herein, “delivering” may include various means of guiding and/or providing therapy to the one or more regions of interest, such as conveying therapy to the one or more regions of interest or directing therapy towards the one or more regions of interest. As will be appreciated, in certain embodiments the delivery of therapy, such as RF ablation, may necessitate physical contact with the one or more regions of interest requiring therapy. However, in certain other embodiments, the delivery of therapy, such as high intensity focused ultrasound (HIFU) energy, may not require physical contact with the one or more regions of interest requiring therapy.

The system 10 may also include a medical imaging system 18, which may comprise an ultrasound control system, that is in operative association with the catheter 14 and configured to image one or more regions of interest. The imaging system 10 may also be configured to provide feedback for therapy delivered by the catheter or separate therapy device (not shown). Accordingly, in one embodiment, the medical imaging system 18 may be configured to provide control signals to the catheter 14 to excite a therapy component of the imaging and therapy transducer and deliver therapy to the one or more regions of interest. In addition, the medical imaging system 18 may be configured to acquire image data representative of the anatomical region of the patient 12 via the catheter 14.

As illustrated in FIG. 1, the imaging system 18 may include a display area 20 and a user interface area 22. However, in certain embodiments, such as in a touch screen, the display area 20 and the user interface area 22 may overlap. Also, in some embodiments, the display area 20 and the user interface area 22 may include a common area. In accordance with aspects of the present technique, the display area 20 of the medical imaging system 18 may be configured to display an image generated by the medical imaging system 18 based on the image data acquired via the catheter 14. Additionally, the display area 20 may be configured to aid the user in defining and visualizing a user-defined therapy pathway. It should be noted that the display area 20 may include a three-dimensional display area. In one embodiment, the three-dimensional display may be configured to aid in identifying and visualizing three-dimensional shapes. It should be noted that the display area 20 and respective controls could be remote from the patient, for example a control station and a boom display disposed over the patient.

Further, the user interface area 22 of the medical imaging system 18 may include a human interface device (not shown) configured to facilitate the user in identifying the one or more regions of interest for delivering therapy using the image of the anatomical region displayed on the display area 20. The human interface device may include a mouse-type device, a trackball, a joystick, a stylus, or a touch screen configured to facilitate the user to identify the one or more regions of interest requiring therapy for display on the display area 20.

As depicted in FIG. 1, the system 10 may include an optional catheter positioning system 24 configured to reposition the catheter 14 within the patient 12 in response to input from the user. The catheter positioning system 24 may be of any type known in the art, or disclosed in the parent application, U.S. patent application Ser. No. 11/289,926, filed Nov. 30, 2005, which is incorporated by reference for this and for teachings related to the interconnect. Moreover, the system 10 may also include an optional feedback system 26 that is in operative association with the catheter positioning system 24 and the medical imaging system 18. The feedback system 26 may be configured to facilitate communication between the catheter positioning system 24 and the medical imaging system 18.

Referring further to FIG. 1, system 10 further comprises a processor 21 for performing several functions, including but not limited to receiving image data sets obtained by the transducer array, image reconstruction, image processing for display and correction techniques that will be described in greater detail with reference to embodiments of the present invention.

FIG. 2 is an illustration of an exemplary embodiment of a rotating transducer array assembly 30 for use in the imaging system of FIG. 1, which may be incorporated into catheter tips as described herein. As shown, the transducer array assembly 30 comprises a transducer array 32, a micromotor 40 (a type of an actuator), which may be internal or external to the space-critical environment, a drive shaft 38 or other mechanical connections between micromotor 40 and the transducer array 32. The assembly 30 further includes a catheter housing 44 for enclosing the transducer array 32, the micromotor 40, an interconnect 45 and the drive shaft 38. In this embodiment, the transducer array 32 is mounted on drive shaft 38 and the transducer array 32 is rotatable with the drive shaft 38. Further in this embodiment, a motor controller 42 and micromotor 40 control the motion of transducer array 32 for rotating the transducer. In an embodiment, the micromotor 40 is placed in proximity to the transducer array 32 for rotating the transducer array 32 and drive shaft 38 and the motor controller 42 is used to control and send signals to the micromotor 40. Interconnect 45 refers to, for example, cables and other connections coupled between the transducer array 32 and the imaging system 18 shown in FIG. 1 for use in receiving/transmitting signals between the transducer and the imaging system. In an embodiment, interconnect 45 is configured to reduce its respective torque load on the transducer and motion controller due to a rotating motion of the transducer. It is noted that transducer array 32 may be incorporated, as shown in FIG. 2, into a transducer assembly 100, but this arrangement is not meant to be limiting.

Catheter housing 44 is of a material, size and shape adaptable for internal imaging applications and insertion into regions of interest. The catheter housing 44 may be integral, or may be comprised of a catheter tip attachable to a catheter body as described herein. The catheter housing 44 further comprises an acoustic window 46. Acoustic window 46 is provided to allow coupling of acoustic energy from the rotating transducer array 32 to the region or medium of interest. The window 46 and fluid between the window 46 and the transducer array 32 allow efficient transmission of acoustic energy from the array 32, which is inside the transducer array assembly 30, to the outside environment. In some embodiments, the window 46 and the fluid have impedance (acoustic) of about 1.5 MRayls. In an embodiment, the motor controller is internal to the catheter housing as shown in FIG. 2. In another embodiment, the motor controller 42 is external to the catheter housing. It is to be appreciated that micromotors and motor controllers are becoming available in miniaturized configurations that may be applicable to embodiments of the present invention. Micromotor and motor controller dimensions are selected to be compatible with the desired application, for example to fit within the catheter for a particular intracavity or intravascular clinical application. For example, in ICE applications, the catheter housing and components contained therein may be in the range of about 1 mm to about 4 mm in diameter. In certain embodiments, transducer array 32 is adapted to fire a plurality of beams 51-5n in order to generate a 3D imaged volume 50.

Various embodiments of ultrasound probe catheter tips comprise a cylindrical outer capsule, such as a plastic outer housing, within which are arranged a more distally positioned electromechanical actuator connected by a drive shaft to a more proximally positioned transducer array, which is connected to an interconnect adapted to communicate with an imaging or therapy system. However, this arrangement is not meant to be limiting, and other arrangements exist for components within a catheter tip embodiment of the invention. In order to eliminate air bubbles that may interfere with ultrasonic imaging, and in order to maintain a desired acceptable temperature of the probe and the transducer array, a number of approaches are employed. Some of these approaches involve fluid passage through both the catheter tip and a catheter body to which it is attached, thereby providing a catheter system.

In various embodiments, an actuator, such as an electromechanical actuator, is positioned more distal than a transducer array that it moves, thus eliminating such drive shaft through the catheter body. This arrangement, generally depicted in FIG. 2 for any type of actuator, allows more space for an interconnect that delivers signals and receives data from the transducer array, but embodiments are not to be limited by this arrangement. Other motor-transducer arrangements are envisaged.

In accordance with an aspect of the present invention, an ultrasonic diagnostic imaging system for acquiring three-dimensional (3D) image data sets is provided. The system comprises a transducer array, as described above, adapted to obtain a plurality of 3D image data sets along a given range of motion and a processor coupled to the transducer array adapted to receive image data sets from the transducer array and to correct for spatially varying error induced by motion of the transducer array. As used herein, the term “spatially varying error” shall refer to the error induced by the motion of the transducer array when the error is not constant from one position of the transducer array to subsequent positions.

Further, in accordance with another aspect of the present invention, a method for ultrasonic diagnostic imaging is provided. The method comprises obtaining a plurality of three-dimensional (3D) image data sets along a given range of motion and/or a plurality of firing positions and correcting the plurality of 3D image data sets for spatially varying error induced by motion of the transducer array.

As described above, a reduction of the error between the desired position and actual position of the transducer array can improve overall image quality. Reducing the error implies that the beam or beam set, when fired, is acquiring the data at the location the system is expecting, thereby reducing image jitter and geometric distortion. In accordance with aspects of the present invention, several methods may be employed to achieve this error reduction and the methods can be divided into three categories: 1) modifying the mechanical system components; 2) employing transducer array position information to correct for error induced by motion of the transducer array; and, 3) modifying timing of beam firings of the transducer array. Each will be described in greater detail below.

In a first embodiment, the mechanical system components and/or system dynamics may be modified. In this approach, the method of reducing the error is through modifications of the mechanical system components or the environment in which it is operating. Referring once again to FIG. 2, the mechanical drive system comprised of motor 40, motor controller 42 and drive shaft 38 may utilize a three-phase open-loop-controlled motor and a gear head (not shown) to drive an ultrasound array. The gear head can introduce both backlash and compliance in the system, which can lead to errors between the commanded position of the array and the actual position of the array. It may be possible to replace the gear head with a zero backlash and stiff gear train and/or introduce a viscous fluid in the operating environment. These modifications serve to reduce the natural dynamics of the drive system and can reduce errors between actual and desired position of the array. In another embodiment for mechanical system correction, it may be possible to reduce the tracking error by implementing a closed loop feedback motion control system. With position sensors on the array and/or on the motor, a feedback control system, such as a proportional-integral-derivative (PID) controller could reduce the errors between the actual and assumed position of the array by dynamically modifying the motor drive signals (using the feedback information to fix the positioning of the motor and the array)

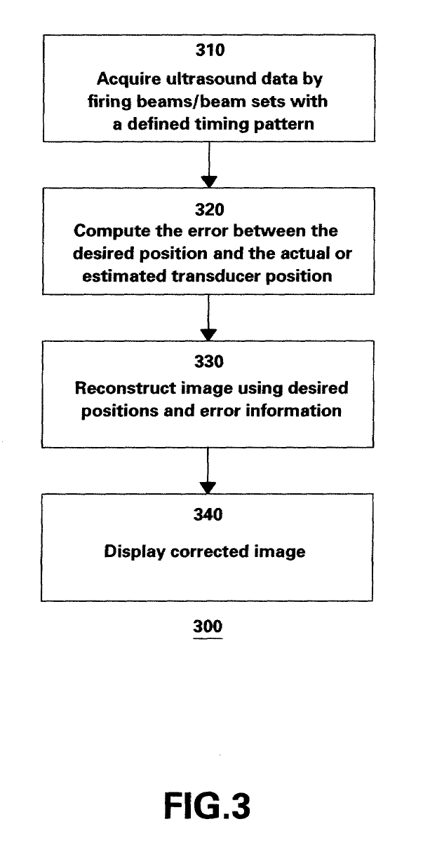

In a second embodiment, transducer array position information may be employed to correct for error induced by motion of the transducer array. In this embodiment, the image reconstruction system, for example contained within processor 21 of FIG. 1, is provided with the array position for each beam (see beams 51-5n, FIG. 2) or beam set fired. The beams may be programmed to fire at evenly spaced points in time, or with some other firing pattern in time, for a given image or volume. However, there may be errors between the desired position and the actual position of the transducer at the time of firing. Therefore, if the position of the array is known when each beam or beam set is fired, the reconstruction of the ultrasound image or volume can be easily created. For the display, the data can be smoothed and or interpolated if necessary. Referring to FIG. 3, an exemplary embodiment 300 for error correction, the first step 310 in the method is to acquire an image data set based on a desired beam set firing pattern in time. Step 320 is to compute the error between the desired and actual (or estimated) position of the transducer when the beam set is fired. The error in step 320 may be calculated in one of several ways, details are covered in the following paragraphs. In step 330, the ultrasound image is reconstructed using the desired position for a given beam set and the error information. Equivalently, the ultrasound image is reconstructed using the actual or estimated position of the transducer array when the beam set is fired. Then, in step 340, the corrected image is displayed.

In an embodiment of step 320 (computing the positional error in the actual transducer trajectory), one method of estimating the position of the array is to use a differential-equation-based model of the array drive system dynamics. The simulation could run before acquisition of an image or volume set. The parameters of the array and drive system, such as inertias, load compliance, backlash, friction, and damping could be used in conjunction with the operational parameters for the current image or volume set. The operational parameters may include scan angle, image depth, and ultrasound beam density required for sufficient ultrasound image contrast and resolution. For example, the scan angle, image depth, volume rate and beam density are specified and used to create a motor trajectory. The motor trajectory is input into the dynamic system model or simulation and the estimated transducer position is calculated. The error between the simulated position of the array and the computed desired trajectory of the array could be used in the manner described above to reconstruct the images.

In another embodiment of step 320, a second method of estimating the position of the array uses a parametric model of the system. A parametric model is again based on parameters of the drive system but the position of the array is easily calculated with linear, non-linear, and trigonometric functions. No iterative solution is necessary. While the estimated array position can be quickly pre-calculated for the given operating conditions, it may not capture all of the complex motion exhibited by the array. In the simplest case, the parametric model may account for backlash in the gearbox or drive mechanism by applying a constant offset or shift to each volume data set depending only on the direction of acquisition. In a slightly more complicated embodiment, the parametric model could be a simple linear model dependent on the scan angle to account for errors introduced by the compliance of the gearbox. In this way, as the loads increase with large scan angles, the parametric model would predict an increasing error between the scan angle commanded by the motor controller and the actual scan angle achieved by the transducer array. Again, the error between the calculated position of the array and the computed desired trajectory of the array could be used in the manner described above to reconstruct the images. The ultimate full extension of the parametric motion compensation above is a full dynamic model used to calculate the transducer position errors that are in turn used to correct the image reconstruction. This dynamic parametric model could be based on a full physics model of the motor, drive system, and transducer, or it could be derived from measured data.

In a third embodiment for step 320, the method calculates the error based on actual measured data of the array position. The actual array position could be measured as the system drives the array through a given motion trajectory. The measured data could be taken for a discrete set of operating conditions, perhaps at time of manufacture or just prior to use, and stored with the ultrasound probe. In this case the position sensing system could be an additional component used in conjunction with the probe, essentially a calibration device. During normal operation, if the stored position data does not cover all operating conditions, interpolation of the data could be used to estimate the position of the array. The measurements and the computed error could be used in the manner described above to reconstruct the images.

In a fourth embodiment for step 320, it may be possible to have a sensor system that is integrated into the probe and allows for real-time measurement of the array position. The measurements and the calculated error could be used just prior to an initiated scanning or could be utilized continuously to reconstruct the image for each beam set.

In a third embodiment, correction for position errors due to motion is performed by modifying the timing of beam firings of the transducer array. In this embodiment, the processor may be configured to provide signals to modify timing of beam firing from the transducer array to correct for error induced by motion of the transducer array. In this approach, one can assume that the imaging system is expecting the ultrasound data that are used to construct the image to be acquired from ultrasound beams that are arranged in some regular geometric pattern, thus the firing of the beams should occur at the appropriate time to match the geometric pattern during the motion of the array. If the array position is known or can be estimated, utilizing methods described above (i.e., differential equation model, parametric model, pre-measured calibration, or integrated position sensor) the position information can be used to determine the correct moment to fire the ultrasound beam such that the data acquired conforms to the expected geometric spacing for the image reconstruction algorithm. For example, assume the ultrasound data are expected by the image reconstruction algorithm at evenly spaced positions. The motion control system could then develop an array trajectory based on desired operating conditions (e.g., scan angle, volume rate). The time to fire beams would be based on evenly spaced points in the trajectory. However, if the actual position of the array does not perfectly track the desired trajectory, then to acquire ultrasound data at evenly spaced intervals in position, the timing of the firings of the beams would be adjusted based on the error data. Desirably, in a further embodiment, the error information can be used to locate the position on the drive trajectory that corresponds to the correct actual firing position. The time for that point on the trajectory can be calculated based on the trajectory profile and stored. Then the beam is fired at the appropriate time to ensure that the data are acquired at evenly spaced intervals with respect to the array position (and not necessarily evenly spaced with respect to time).

Since ultrasound requires a finite time for propagation, it may be necessary, when using this embodiment, to reduce the overall imaging rate or to fire ultrasound beams that overlap in time or to reduce the number of beams fired. If the desired trajectory is designed for continuous ultrasound imaging at a uniform beam firing rate, then deviations from that trajectory will increase the time interval between some beams and reduce the time interval between other beams. If the reduced time is less than the time required for ultrasound propagation to and from the desired imaging depth, then beams will overlap in time or the imaging rate must be reduced or some beams must be skipped.

In the case of creating successive 3D images of the same anatomy by reciprocating motion, errors in the mechanical sweeping motion can cause differences between successive images, known as image jitter. One known approach is to shift the planned time for all firings of an ultrasound beam/beam set. The shift is a constant timing offset that can be applied to every other image, to align the image acquired in one direction of motion with the image acquired in the other direction of motion. Alternatively, the timing offset can be divided, with one offset applied during one direction of motion and another offset applied during the other direction of motion. The constant timing offset can be used to reduce image jitter caused by backlash in the mechanical system. The methods discussed below allow for more than a constant offset in timing, and thus provide methods for further reducing the image jitter and geometric distortion associated with mechanically oscillating transducers.

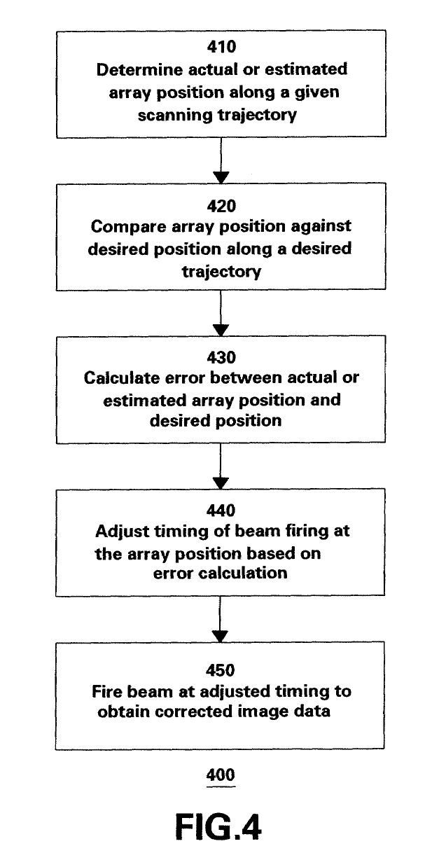

Referring to FIG. 4, a method 400 for correcting for motion by modifying beam firing timing is shown. In step 410, the array position is either determined or estimated (hereinafter “actual/estimated position”). Details for estimating array position will be described in greater detail below. At step 420, during acquisition the estimated, interpolated, or known array position along a given scanning trajectory is compared against the desired scanning trajectory. At step 430, the error between the actual/estimated position and desired position is calculated. At step 440, beam firing timing is adjusted based on error calculated at step 430. Finally, at step 450 the beam is fired using the adjusted timing such that the beam is aligned with the desired position along a given scanning trajectory, and thereafter the image data is obtained.

In an embodiment of step 410 (estimating the position of the transducer array), one method of estimating the position of the array is to use a differential-equation-based model of the array drive system dynamics. The simulation could run before acquisition of an image or volume set. The parameters of the array and drive system, such as inertias, load compliance, backlash, friction, and damping could be used in conjunction with the operational parameters for the current image or volume set. The operational parameters may include scan angle, image depth, and ultrasound beam density required for sufficient ultrasound image contrast and resolution. The error between the simulated position of the array and the computed desired trajectory of the array could be used in the manner described above to modify the timing of the firing of beams or beam sets.

In another embodiment of step 410, a second method of estimating the position of the array uses a parametric model of the system. A parametric model is again based on parameters of the drive system but the position of the array is easily calculated with linear, non-linear, and trigonometric functions. No iterative solution is necessary. While the estimated array position can be quickly pre-calculated for the given operating conditions, it may not capture all of the complex motion exhibited by the array. Again, the error between the calculated position of the array and the computed desired trajectory of the array could be used in the manner described above to modify the timing of the firing of beams or beam sets.

In a third embodiment for step 410, the method calculates the error based on actual measured data of the array position. The actual array position could be measured as the system drives the array through a given motion trajectory. The measured data could be taken for a discrete set of operating conditions, perhaps at time of manufacture or just prior to use, and stored with the ultrasound probe. In this case the position sensing system could be an additional component used in conjunction with the probe, essentially a calibration device. During normal operation, if the stored position data does not cover all operating conditions, interpolation of the data could be used to estimate the position of the array.

In a fourth embodiment for step 410, it may be possible to have a sensor system that is integrated into the probe and allows for real-time measurement of the array position. The measurements and the calculated error could be used just prior to an initiated scanning or could be utilized continuously to adjust timing of the beam firing during operation of the probe.

In a fourth embodiment, the measured or estimated transducer array position information may be employed to correct for error in the motion of the transducer array. In this embodiment it is assumed that there is a known relationship between the motor drive trajectory and the actual motion of the array. This relationship can be a simple parametric model or a complex differential equation based model of the system, similar to the methods described above. However, in this case, the inverse relationship must be known or be computed, i.e., so that measured or estimated errors in the array position are used to create a modified scanning trajectory for the motor. The new trajectory for the motor is then implemented and the trajectory of the transducer should more closely match the desired position of the transducer.

Any of the four embodiments described above may be used alone, or two or more embodiments may be used in combination. For example, the fourth embodiment (motion correction) may be used to reduce gross errors in the motion trajectory, then the second embodiment (correction during image reconstruction) may be used to mitigate the effects of any remaining motion errors.

In a further aspect of the invention, another embodiment for correcting error due to mechanical motion is provided in which the motion of the transducer array is controlled such that the effects of inherent motion errors on the resulting images are mitigated. In this embodiment, the transducer array is further adapted to image in one direction at a first rate of motion for obtaining the image data sets and adapted to return the transducer array to a starting point at a second rate of motion corresponding to a maximum rate of motion permitted by the at least one motion control device. Small mechanical probes, e.g. 4D intracardiac echocardiography (ICE) catheters, typically have “soft” (in other words, elastic or compliant) low-power drive systems with very repeatable but highly non-linear, asymmetric motion. Imaging during bi-directional motion results in severe image jitter, because the forward images are not aligned with the reverse images. To achieve fast, stable real-time imaging, it may be desirable to image while moving in one direction, then return the transducer array quickly to its original position without imaging.

If the primary goals are speed (volume image update rate) and image stability, and the mechanical drive system is highly repeatable, then one method of achieving these goals is to image while moving in one direction at the fastest rate that the ultrasound will allow: image volume*ultrasound beam density*2 [round-trip]/sound speed/multi-line ratio=minimum time per volume. Upon completion of one imaged volume, it is desirable to return in the opposite direction at the fastest rate that the motor drive system will permit in order to minimize “dead” time and prepare for the next imaging cycle. Motion during imaging may be nominally at constant velocity, if the image beams or frames are uniformly spaced, or the velocity may vary, e.g. as 1/cos(theta) if the image frames are spaced at equal intervals of sin(theta). While moving in the imaging direction, at the endpoints of the range of motion, some time and distance will be required for acceleration and deceleration. If the imaging system can accommodate image data acquired at non-uniform times or spacings, then the acceleration & deceleration time may be used for imaging. If not, then maximum acceleration & deceleration rates should be used, to minimize the non-imaging “dead” time in each 4D imaging cycle. Motion during the fast return will be determined by the maximum acceleration and perhaps max velocity that the motor, gearbox, and mechanical system can achieve and sustain over the desired operating life of the catheter. If the mechanical motion is highly repeatable, then one-way imaging will produce stable images, with minimal volume-to-volume jitter. Any non-linearity in the mechanical motion may cause geometric distortion in the image. First-order non-linearities, e.g. backlash and compliance in a gearbox, may be similar for all assemblies of a given type or lot and may be easily compensated in the motion control or imaging software, using methods described in the above embodiments. Second-order non-linearities, including unit-to-unit variations, typically do not cause significant image distortions, although they would cause significant image jitter if bi-directional imaging were attempted.

In accordance with aspects of the present invention, one-way imaging with fast return may allow for images with greater stability at a higher volume rate than two-way imaging with beam timing adjustment to reduce (but not eliminate) image jitter. The beam timing adjustment, coupled with the finite time required for ultrasound propagation and image acquisition, slows the two-way motion more than the fast return slows the one-way imaging. Imaging in only one direction eliminates the requirement that image planes acquired during forward motion be aligned with image planes acquired during reverse motion, thereby significantly simplifying the system. Complications which must be addressed for bi-directional imaging include motor-to-motor variability; load variability; backlash, compliance, and other nonlinearities in the mechanical drive system; detailed calibration, compensation, or correction of asymmetric motion; non-uniform image acquisition to compensate for non-uniform motion; and image processing to compensate for forward/reverse asymmetries.

It is to be appreciated that restricting the imaging direction of the transducer array (one-way imaging) enables good quality, stable, fast 4D imaging with small, simple, low-cost mechanical components and manufacturing techniques. It requires only that each catheter have transducer motion that is highly repeatable in the short term. A stiff, linear, symmetric drive system is not required. Calibration or compensation of each catheter or of catheter-to-catheter variations is not required. Position or motion sensors are not required. Volume imaging rates are optimized, with non-imaging “dead” time minimized, for the best real-time imaging of moving anatomy, such as the heart.

It should be understood that for some physical systems, applying the correction methods detailed above (image reconstruction adjustments or beam firing adjustments or drive trajectory adjustments based on known, interpolated, or calculated errors) may yield higher overall volume rates and acceptable image stability as compared to the one way imaging approach.

While only certain features of the invention have been illustrated and described herein, many modifications and changes will occur to those skilled in the art. It is, therefore, to be understood that the appended claims are intended to cover all such modifications and changes as fall within the true spirit of the invention.

1. An ultrasonic imaging system for acquiring three-dimensional (3D) image data sets comprising:
a transducer array with a given range of motion adapted to obtain a plurality of 3D image data sets of a region of interest;
a processor coupled to the transducer array adapted to receive image data sets from the transducer array and to correct for spatially varying error induced by motion of the transducer array.

2. The system of claim 1, wherein the transducer array is adapted to scan along the given circumferential field of motion by mechanical motion actuated by at least one motion control device coupled to the transducer array.

3. The system of claim 2, wherein the mechanical motion comprises rotating motion, oscillating motion, and a combination thereof.

4. The system of claim 2, wherein the motion control device comprises a motor controller coupled to each of a drive shaft and motor to control motion of the transducer array motor.

5. The system of claim 1, wherein the transducer array is enclosed within a catheter assembly and the catheter assembly comprises:
at least one motion control device coupled to the transducer array for controlling motion of the transducer array during acquisition of image data sets.

6. The system of claim 4, wherein the processor is adapted to obtain position error information for at least one position of the transducer array along the given range of motion during acquiring of the image data sets and wherein the position error information is obtained from at least one of a differential-equation-based model of drive mechanisms in the motion control device, a parametric model of the drive mechanisms or actual measured data of the array position.

7. The system of claim 6, wherein the processor is adapted to correct for the position error information during reconstruction of the image data sets.

8. The system of claim 6, wherein the processor is further adapted to store predetermined position error information for at least one of various operating modes/conditions, various operating environments, various ranges of motion and combinations thereof.

9. The system of claim 8, wherein the transducer array further comprises at least one sensor for measuring a position of the transducer array and the position error measurements are obtained concurrently during acquisition.

10. The system of claim 1, wherein the processor is configured to enable modifying timing of beam firing from the transducer array to correct for error induced by motion of the transducer array.

11. The system of claim 6, wherein the processor is further configured to modify motor control signals using the position error information to reduce error between an actual position and a desired position of the transducer array.

12. The system of claim 1, wherein the processor is configured to perform the following steps:
determining an actual or estimated transducer array position at each position along a given scanning trajectory;
comparing the actual or estimated array position at each position along the given scanning trajectory against a corresponding desired position along a desired trajectory;
calculating an error between the actual or estimated position and desired position; and,
adjusting beam firing timing of the transducer array along the trajectory based on the error calculation.

13. The system of claim 2 wherein the transducer array is further adapted to image in one direction at a first rate of motion for obtaining the image data sets and adapted to return the transducer array to a starting point at a second rate of motion corresponding to a higher rate of motion permitted by the at least one motion control device.

14. A method for ultrasonic diagnostic imaging using a mechanically moving transducer array, the method comprising:
obtaining a plurality of three-dimensional (3D) image data sets along a given range of motion and/or a plurality of firing positions; and,
correcting for spatially varying errors induced by motion of the transducer array in order to display 3D image data sets.

15. The method of claim 14, further comprising measuring and storing a plurality of predetermined position error measurements for the transducer array for at least one of various operating modes/conditions, various operating environments, various ranges of motion and a combination thereof

16. The method of claim 14, wherein the transducer array further comprises at least one sensor for measuring an actual position of the transducer array and error measurements for the actual position are obtained concurrently during acquisition.

17. The method of claim 14, wherein the correcting for errors comprises obtaining position error information for at least one position of the transducer array along the given range of motion during acquiring of the image data sets and wherein the position error information is obtained from at least one of a differential-equation-based model of drive mechanisms in a motion control device driving the transducer array, a parametric model of the drive mechanisms or actual measured data of the array position.

18. The method of claim 17, wherein the correcting step comprises enabling modifying timing of beam firing from the transducer array to correct for error induced by motion of the transducer array.

19. The method of claim 18, wherein the correcting step comprises:
determining an actual or estimated array position at each position along a given scanning trajectory;
comparing the actual or estimated array position at each position along the given scanning trajectory against a corresponding desired position along a desired trajectory;
calculating an error between the actual/estimated position and desired position; and,
adjusting beam firing timing of the transducer array along the trajectory based on the error calculation.

20. The method of claim 17, further comprising using the position error information during reconstruction of image data sets.

21. The method of claim 16, wherein the transducer array is adapted to scan along the given circumferential field of motion by mechanical motion actuated by at least one motion control device coupled to the transducer array; and,
wherein the transducer array is further adapted to image in one direction at a first rate of motion for obtaining the image data sets and adapted to return the transducer array to a starting point at a second rate of motion corresponding to a higher rate of motion permitted by the at least one motion control device.

22. The method of claim 17, wherein the correcting for errors further comprises modifying motor control signals of the motion control device using the position error information to reduce error between an actual position and a desired position of the transducer array.
